(19) United States
(12) Patent Application Publication
(10) Pub. No.: US 2025/0000828 A1
(43) Pub. Date: Jan. 2, 2025

(54) WOUND CARE AND METHOD FOR PREVENTING WOUND INFECTION

(71) Applicant: EPIEN Medical, Inc., St. Louis Park, MN (US)

(72) Inventor: Steven J. Kavros, Waconia, MN (US)

(21) Appl. No.: 18/361,283

(22) Filed: Jul. 28, 2023

Related U.S. Application Data
(60) Provisional application No. 63/510,734, filed on Jun. 28, 2023.

(51) Int. Cl. A61K 31/185; A61B 17/32; A61N 1/32; A61N 5/06; A61N 5/067; A61N 7/00; A61P 17/02

(52) U.S. Cl. CPC A61K 31/185; A61B 17/32; A61N 1/326; A61N 5/0616; A61N 5/067; A61N 7/00; A61P 17/02; A61B 2017/320004; A61N 2007/0017

(57) ABSTRACT

Embodiments include a method of promoting healing of tissue that has been wounded, comprising: applying a formulation that includes phenolsulfonic acid and either guaiacolsulfonic acid or benzenesulfonic acid to tissue that has been wounded; normalizing pH of the tissue that has been wounded; and re-applying the formulation that includes phenolsulfonic acid and either guaiacolsulfonic acid or benzenesulfonic acid to the tissue that has been wounded over a time period effective to close a cut or break in the tissue that has been wounded and accelerating the healing the tissue that has been wounded compared to wounded tissue that has not been treated with the formulation that includes phenolsulfonic acid and either guaiacolsulfonic acid or benzenesulfonic acid.

### CROSS-REFERENCE TO RELATED APPLICATION

This patent application claims the benefit of U.S. Provisional Patent Application No. 63/410,734, filed Jun. 28, 2023, entitled “WOUND CARE ANDI METHOD FOR PREVENTING WOUND INFECTION”, which is incorporated by reference herein in its entirety.

### FIELD

Inventive embodiments disclosed herein include method embodiments for promoting healing of tissue that has been wounded.

### BACKGROUND

Approximately 2.5% of the United States (US) population suffers from chronic wounds, which cost Medicare nearly $97 billion a year. A majority of chronic wounds (70%) are venous leg ulcers (VLUs). However, 75% of diabetic foot ulcers (DFUs) remain unhealed at 12 weeks, and diabetic foot disease alone is costing the US economy $80 billion. Chronic wounds continue to be a source of significant morbidity and, tragically, mortality worldwide. High recurrence rates of VLUs (70% at 10 years) and DFUs (65% at 3 years) are reported. Twenty percent of patients with DFUs undergo an amputation (the incidence of which is increasing by 50% among vulnerable populations); and 50%-70% of patients with DFUs die within 5-years.

It is imperative that a clinician treating wounds understand the impact and importance of wound bed preparation as it relates to healing. There are many molecular and chemical factors to consider to effectively manage the patient through the process of resetting the chronicity in a wound that has persisted over 30 days to an acute state. For example, metalloproteinases (MMPs) are essential to tissue remodeling, but when they are left unregulated in the wound bed, they degrade the restored extracellular matrix.

Debridement plays a critical role in wound bed preparation and facilitates the regulation of MMPs and other proinflammatory proteases that are elevated in chronic wounds. In addition to removing necrotic tissue and senescent cells, debridement can eliminate bacteria that are frequently harbored within the tissue. Sharp debridement is the gold standard method for removing infected and devitalized tissue to allow the wound to progress to a normal healing state, but no method of debridement is 100% effective, especially as foreign bodies can return to the wound after initial debridement occurs.

Chronic wounds, that is, wounds that persist for over 30 days, have multiple additional molecular and chemical factors that delay or stall a normal linear healing trajectory. The problem of wound chronicity negatively effects quality of life, patient outcomes, and is a significant burden to healthcare economic system. It is well documented that the alkaline pH of the wound bed is one of the factors that inhibit wound healing. Normal pH of the human dermis is age dependent, however, the generally accepted range is 4.8-6.2.

### Summary

Embodiments disclosed herein include a method of promoting healing of tissue that has been wounded, comprising applying a formulation that includes phenolsulfonic acid and either guaiacolsulfonic acid or benzenesulfonic acid to tissue that has been wounded; normalizing pH of the tissue that has been wounded; and re-applying the formulation that includes phenolsulfonic acid and either guaiacolsulfonic acid or benzenesulfonic acid to the tissue that has been wounded over a time period effective to close a cut or break in the tissue that has been wounded and accelerating the healing the tissue that has been wounded compared to wounded tissue that has not been treated with the formulation that includes phenolsulfonic acid and either guaiacolsulfonic acid or benzenesulfonic acid.

Embodiments also include a method of promoting healing of tissue of a surgical site including an incision portion, comprising: normalizing pH of an environment contacting tissue of the surgical site by contacting the tissue including the incision with a formulation that includes sulfonic acid and either guaiacolsulfonic acid or benzenesulfonic acid until the incision closes.

### DESCRIPTION OF DRAWINGS

FIG. 1 is a graphical view of mean pH values measured throughout the course of the study for all wounds for patients treated with regenerative debridement technology, RDT, disclosed herein and wounds not treated with the RDT, identified as a Control Group. Note that the control group did not have a pH measurement taken on Day 0 post application.

FIG. 2 is a graphical view of mean pH values measured throughout the course of the study for diabetic foot ulcers (DFUs) for ulcers treated with RDT and ulcers not treated with RDT, identified as the Control Group. Note that the Control group did not have a pH measurement taken on Day 0 post application.

### DETAILED DESCRIPTION

Embodiments of the invention are described in sufficient detail to enable those skilled in the art to practice the invention Other embodiments may be utilized and structural, logical, chemical, and other changes may be made without departing from the spirit or scope of the invention discussed herein. The following detailed description is, therefore, not to be taken in a limiting sense, and the scope of the inventive embodiments discussed herein is defined only by the appended claims.

Inventive embodiments disclosed herein include a method of promoting healing of tissue that has been wounded, comprising, normalizing pH of the tissue that has been wounded, by application of a Regenerative Debridement Technology, hereinafter RDT, formulation that includes phenolsulfonic acid and either guaiacolsulfonic acid or benzenesulfonic acid and; and re-applying the RDT formulation that includes phenolsulfonic acid and either guaiacolsulfonic acid or benzenesulfonic acid over a time period effective to close the cut or break in the tissue that has been wounded and accelerating the healing the tissue that has been wounded compared to wounded tissue that has not been treated with the formulation that includes phenolsulfonic acid and either guaiacolsulfonic acid or benzenesulfonic acid.

Inventive embodiments also include a method of promoting healing of tissue of a surgical site, including an incision, that includes normalizing pH of tissue of the surgical site, including the incision, by contacting the tissue with a formulation that includes sulfonic acid and phenolsulfonic acid or guaiacolsulfonic acid and normalizing the pH of the environment until the incision closes.

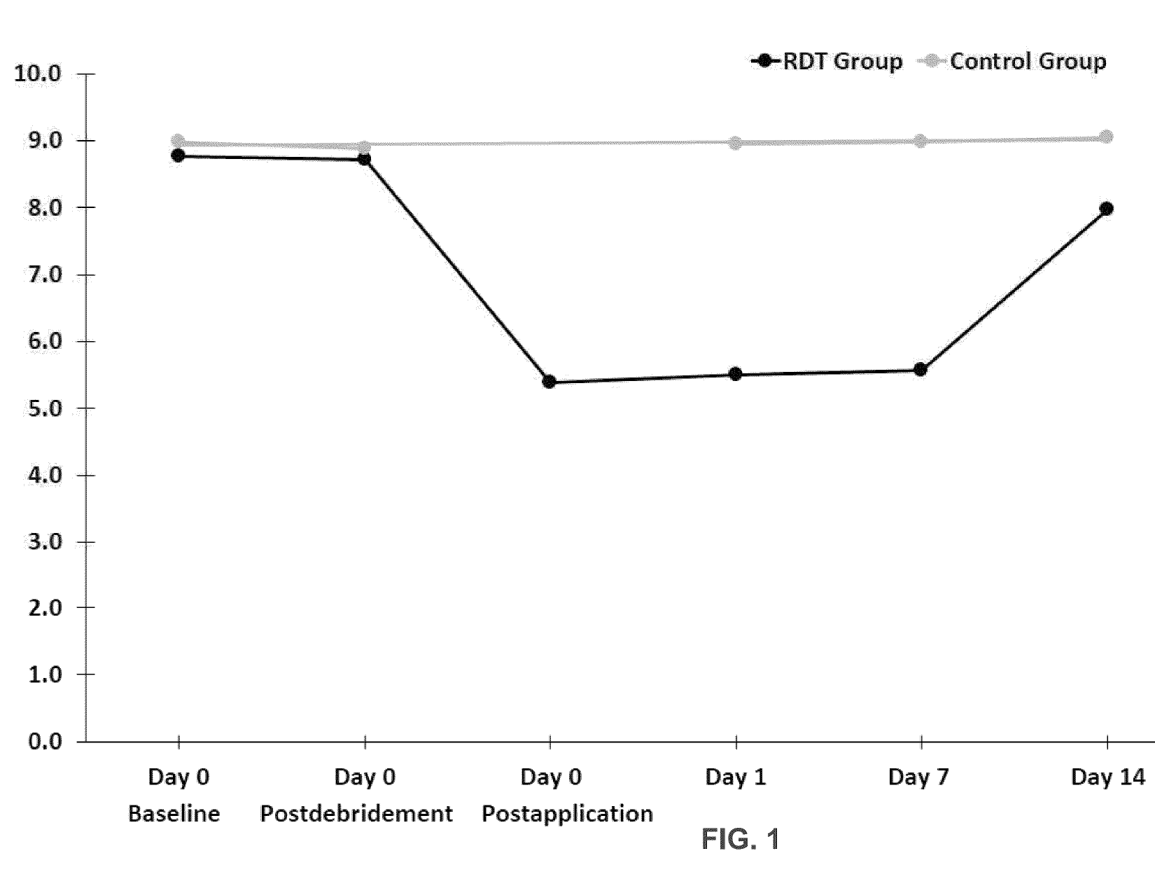

A breakthrough, nonbiologic desiccant technology has been developed as a debridement agent, described herein as Regenerative Debridement Technology or RDT. RDT includes a topical liquid that is applied to a wound bed, resulting in an intense water extraction harboring necrotic debris and senescent cells, bacteria, yeast, fungi, and proinflammatory factors. These unwanted factors are concentrated in a coagulum RDT applied to a chronic, alkaline wound bed imparts a normal physiologic dermal pH for at least one week's duration as shown in FIG. 1.

Modulating and normalizing wound pH allows for an improved pathway in wound healing generally and chronic wound healing, in particular, as further evidenced herein by rapid closure rates at 2 weeks compared to control wounds not treated with RDT RDT is a topical liquid agent for use by healthcare practitioners in the cleansing of lesions of the skin and damaged dermal tissue during standard mechanical debridement procedures. It removes necrotic tissue and destroys biofilm upon contact, and reduced proinflammatory markers that become unbalanced in chronic wounds. This chemical process works on a unique non-biologic platform; mechanical, cellular, and molecular. Mechanism of action centers around rapid extraction of water from the wound bed forming a coagulum that harbors the necrotic tissue and senescent cells, biofilm, and proinflammatory factors. This rapid desiccation process takes less than a minute and is selective to diseased tissue, leaving normal tissue unharmed. Additionally, preclinical studies clearly demonstrate a positive effect on keratinocyte stimulation and thickness, granulation tissue formation, white blood cell migration, and angiogenesis.

One inventive embodiment disclosed herein includes a method of promoting healing of tissue that has been wounded. The method includes desiccating the tissue that has been burned and unburned tissue proximal to the tissue that has been burned, by application of an RDT formulation that includes phenolsulfonic acid or guaiacolsulfonic acid or phenolsulfonic acid and guaiacolsulfonic acid or phenolsulfonic acid and benzenesulfonic acid and normalizing the pH of the burned tissue over a time period as long as seven days so that the pH of the burned tissue is the same as healthy unburned tissue. The pH of the burned tissue is typically normalized from an alkaline environment to a normal dermal pH by removing excessive proinflammatory factors as well as pathologic planktonic bacteria and biofilm. This occurs by a desiccation process of the chemical formulary. In some instances, the application of the RDT normalizes pH by adjusting pH from an acidic environment to a higher pH environment. The process takes less than 60 seconds and lasts up to 7 days. The RDT desiccant is typically applied to burned tissue every 7 days.

The oxygenation of the wound bed is a driving force of healing that prevents infections and promotes angiogenesis and tissue remodeling and repair. Reduced oxygen in the chronic wound bed results in an alkaline environment that delays wound healing and makes the wound more susceptible to infection. The pH measurement represents a concentration of hydrogen ions, with a normal, healthy skin surface having an acidic pH ranging from 4.0 to 6.2 Acute wounds trend towards the neutral state but experience a short alkaline peak before transitioning down to an acidic state, with a 0.6 to 0.9 decrease in pH respectively releasing 50% to 5 times more oxygen into the oxygen-deprived wound bed and reducing proinflammatory protease activity by 80%.

An acidic pH additionally induces macrophage recruitment, which reduces tumor necrosis factor-alpha, and promotes fibroblast proliferation, angiogenesis, and epithelialization, while preventing infection. Untreated chronic wounds, on the contrary, remain in an alkaline state ranging from 7.2 to 8.9, in which proinflammatory proteases, enzymes, and growth factors flourish. Furthermore, debridement, an often necessary component of wound management, also increases a wound's pH. The RDT disclosed herein is a therapeutic target in wound care and has been shown to provide both an immediate and sustained effect. The RDT mitigates factors of debridement that increase a wound's pH.

A nonbiologic desiccant technology has been developed for wound bed preparation following debridement. Regenerative Debridement Technology (RDT) is a topical, therapeutic liquid that, when applied to the wound bed, results in enhanced shear force and tissue coagulation to remove necrotic and pathogenic debris.

In one embodiment, the method also includes a sharp debriding necrotic tissue that has been burned or otherwise wounded and sloughing the necrotic tissue that has been debrided Sharp debridement of the necrotic tissue is done initially at a bedside or operating room.

Typically, sharp debridement is performed once every seven days. The method also includes debriding planktonic bacteria from the tissue that has been burned or otherwise wounded and treating the burn or wound with one or more of low frequency ultrasound, laser energy, and electro-stimulation.

RDT effectively normalizes wound pH for 1 week, resulting in an accelerated healing process. Sharp debridement removes necrotic tissue but cannot remove all the pathologic planktonic bacteria and biofilm. The bacterial and biofilm remain in the burn bed and repopulate in less than four hours. Adjunctive measures are then employed, such as hydrosurgical, low frequency ultrasound and other topical methods are employed.

### Definitions

The term “wound” as used herein refers to an injury to living tissue that cuts or breaks the skin. Wounds include, but are not limited to chronic venous leg ulcers, pressure ulcers, diabetic ulcers, decubitus ulcers, stasis ulcers, dermal ulcers, burns, and pressure ulcers Chronic wounds are characterized by necrotic tissue that delays healing. Acute wounds occur suddenly and progress through known stages of healing. Wounds also include injuries resulting from gun shots or explosions or accidents that cut or break the skin Wounds as used herein also includes “burns.” Burns as used herein includes injuries to skin or other organic tissue caused by heat, radioactivity, electricity, friction or contact with chemicals. Thermal burns occur when some or all of the cells in the skin or other tissues are destroyed by hot liquids, i.e. scalds; hot solids, i.e. contact burns; or flames, i.e. flame burns.

“Healing” is determined by observing normal processes of angiogenesis, granulation tissue and epithelialization. Healing is noted when there is full epithelial coverage without moisture or drainage. Healing occurs when cuts or breaks in skin are closed.

The term, “normalized pH” as used herein refers to a concentration of hydrogen ions, of a normal, healthy skin surface. Typically, a normalized pH ranges from 4.0 to 6.2.

The term, “wound bed” as used herein, refers to a base or floor of a burn, laceration or chronic ulcer.

The term “debridement” as used herein refers to a removal of necrotic or infected skin tissue to help a wound, including a burn, to heal.

The term, “sharp debridement” as used herein, refers to a removal of dead or necrotic tissue or foreign material from and around a wound to expose healthy tissue using a sterile scalpel, scissors or both.

“Hydrosurgical debridement” as used herein refers to a use of a controlled, high pressure fluid jet to cut and remove necrotic debris, contaminants, and bacteria, thereby facilitating a more aggressive debridement. One description of hydrosurgical debridement can be found here: Hydrosurgical Debridement Allows Effective Wound Bed Preparation of Pressure Injuries: A Prospective Case Series—PMC (nib.gov)

“Low Frequency Ultrasound debridement” as used herein refers to a use of an ultrasound device to transmit low-intensity and low-frequency acoustic energy through a constant flow of saline mist More information can be found here. Low-Frequency Ultrasound Debridement in Chronic Wound Healing: A Systematic Review of Current Evidence—PMC (nib.gov)

Specific RDT desiccating formulation embodiments include the following:

### Formulation 1:

Phenolsulfonic acid—36%-37 by weight;
Guaiacolsulfonic acid—23%-24 by weight;
Sulfuric Acid—28%-29 by weight;
Water—10% by weight; and
Colloidal Silica—3% by weight.

### Formulation 2:

Another embodiment includes a liquid formulation for treating chronic wounds. The embodiment includes the following:
Benzenesulfonic acid—43-45% by weight;
Phenolsulfonic acid—38-40% by weight; and
Water—15% by weight;
Colloidal Silica—5% by weight.

### Formulation 3:

Another embodiment includes a liquid formulation for treating wounds. The formulation includes the following components and concentration ranges:
Phenolsulfonic acid up to 75% wt. percent);
Guaiacolsulfonic acid up to 75% wt. percent);
Free Sulfuric Acid up to 30% wt percent);
Water up to 30% wt. percent); and
Colloidal Silica up to −10% wt. percent). The additive concentration of each material adds up to 100% for the total formulation.

### Formulation 4:

Another formulation embodiment for treating wounds, includes the following components and concentration ranges:
Benzenesulfonic acid up to 75% by weight),
Phenolsulfonic acid up to 75% by weight),
Water up to 30% by weight); and
Colloidal Silica up to 10% by weight)

The additive concentration of each ingredient adds up to 100% for the formulation.

Formulation embodiments disclosed herein are used as an adjunctive agent to all other forms of debridement and are compatible with all other forms of debridement. For one embodiment, the formulation is used as an adjunctive rinse following surgical debridement, also known as sharp debridement, which is a conventional form of mechanical debridement used in the treatment of chronic wounds.

Formulation embodiments include liquid and gel embodiments that, when applied to a chronic wound, effectively reduce the size of the wound and, in some instances, heal the chronic wound.

It is believed that the formulations disclosed herein work by debriding the wound and by destroying biofilms and bacteria on and in a skin wound and desiccating and washing away necrotic tissue defining the wound. The mechanism of action is believed to be the following: Application of a formulation embodiment disclosed herein acts as both a mechanical debridement and a chemical debridement. The mechanical debridement action derives from an elevated viscosity and density that allows the formulation to deliver a higher level of mechanical pressure and shear stress across a surface. The chemical debridement derives from a desiccating action of the formulation embodiment which acts to destabilize the cohesion of necrotized tissues and microbial biofilm so that they are more easily carried away by the mechanical action.

Formulation embodiments not only absorb water mechanically, i.e. absorb water that is adhered to microbes and biofilm within and proximal to a wound, but also absorb water that is electrostatically bonded, i.e. hydrogen bonded, with structural components of bacteria and other microbes in wounds, thereby desiccating bacterial structure and destroying bacterial structural integrity. Formulation embodiments also desiccate any residual necrotic tissue.

Once structural integrity is destroyed, the microbes die and the biofilm is disrupted. Bacteria, and other pathogens exposed to formulation embodiments disclosed herein do not develop a resistance to the formulations. Formulation and method embodiments disclosed herein are effective against all bacteria, biofilm, prions and other infectious agents.

Formulation embodiments disclosed herein are exposed to the bacterial and other pathogen structures that are desiccated by formulation embodiments disclosed herein. The structures desiccated include cell wall, plasma membrane, S-layers, glycocalyx, flagella, fimbriae and pili. Structures of both gram negative and gram positive bacteria are desiccated and susceptible to destruction. While bacteria are described, it is understood that all microbes in a biofilm are susceptible to destruction by desiccation by a formulation embodiment disclosed herein.

Additionally, formulation embodiments disclosed herein prevent damage done by a destructive inflammation immune response initiated by necrotizing tissue by eliminating the necrotizing tissue before the response is prompted. Elimination of the necrotic tissue prevents the body from initiating a destructive inflammatory immune response Formulation embodiments disclosed herein are effective for killing bacteria that are antibiotic resistant; such as MRSA. Formulations disclosed herein are also useful in killing bacteria that are in a persistent state. Bacteria in a persistent state are not completely immune to antibiotics but remain inactive or dormant in the presence of antibiotic treatment. Once an antibiotic regime ends, the persistent bacteria resume their activity. Recent research identified the stimulus for putting the bacteria into a persistent state which is attack by an antibiotic.

Formulation embodiments disclosed herein are also believed to kill bacteria that have not been cultured in a lab. Bacteria that have not been cultured in a lab exist in nature but do not grow on laboratory media. At the present time, these bacteria are identified by their unique genetic material. These bacteria are killed by desiccation in the manner disclosed herein.

Formulation embodiments that include free sulfuric acid have the free sulfuric acid component because sulfonation of the phenol and guaiacol or benzene performed does not remove residual sulfuric acid remaining at the conclusion of the sulfonation reactions. In order to drive the sulfonation reaction to completion so that all of the phenol and guaiacol or benzene molecules become sulfonated, excess sulfuric acid is added to a reaction mixture in a ratio of approximately 1:4:1 (phenolsulfonic acid to guaiacolsulfonic acid or benzenesulfonic acid to sulfuric acid so that there is an excess of sulfuric acid when the reaction is completed. The excess sulfuric acid is referred to as “free sulfuric acid” because it is not conjugated to a phenol or guaiacol.

The formulation embodiments that do not contain any free sulfuric acid are blended from a purified sulfonated aromatic that is obtained already in sulfonated form. Any residual or excess free sulfuric acid is removed, so that when blending is completed, there is substantially zero free sulfuric acid in the blend.

Specific liquid and gel formulations usable for treating wounds include the following:

### Formulation 5: Contains Free Acid in a Gel Form

Phenolsulfonic acid 36% by weight
Guaiacolsulfonic acid—23% by weight
Free Sulfuric Acid—28% by weight
Water—10% by weight
Colloidal Silica—3% by weight
   Formulation 6: Contains no free acid, in a liquid form
Benzenesulfonic acid—45% by weight
Phenolsulfonic acid—40% by weight
Water—15% by weight
   Formulation 7: Contains no free acid, in a gel form
Benzenesulfonic acid—43% by weight
Phenolsulfonic acid—38% by weight
Water—14% by weight
Colloidal Silica—5% by weight
Other formulation embodiments are disclosed below:

### Formulation 8

| Component | Concentration Ranges | |
|---|---|---|
| Phenolsulfonic Acid | 25-80% | by weight |
| Guaiacolsulfonic Acid | 25-80% | by weight |
| Ammonium Phenolsulfonate | 0-32% | by weight |
| Water | 13-30% | by weight |

The additive concentration of each ingredient adds up to 100% for the formulation

### Formulation 9:

| Component | Concentration Ranges | |
|---|---|---|
| Phenolsulfonic Acid | 25-80% | by weight |
| Guaiacolsulfonic Acid | 25-80% | by weight |
| Ammonium Phenolsulfonate | 0-5% | by weight |
| Water | 13-30% | by weight |

The formulation is applicable to the wound as a liquid or as a gel. The additive concentration of each ingredient adds up to 100% for the formulation.

While specific formulations have been disclosed, it has been found that formulations having the following ranges are also suitable for use:

Formulation 10 containing Free Acid (percent by weight)
Phenolsufonic acid (0-75% by weight)
Guaiacolsulfonic acid (0-75% by weight)
Free Sulfuric Acid (0-30% by weight)
Water (0-30% by weight)
Colloidal Silica (0-10% by weight)

The additive concentration of each ingredient adds up to 100% for the formulation Formulation 11 with no Free Acid (percent by weight)
Benzenesulfonic acid (0-75% by weight)
Phenolsulfonic acid (0-75% by weight)
Water (0-30% by weight)
Colloidal Silica (0-10% by weight)

The additive concentration of each ingredient adds up to 100% for the formulation.

Other embodiments include method embodiments for treating wounds. Wounds include chronic wounds include, but are not limited to chronic venous leg ulcers, pressure ulcers, diabetic ulcers, decubitus ulcers, stasis ulcers, dermal ulcers, burns, and pressure ulcers. Chronic wounds are characterized by necrotic tissue that delays healing.

The method embodiments include applying a formulation embodiment disclosed herein to the wound for 5 to 60 seconds. For some embodiments, a first debriding, such as mechanical debriding, is performed before adding the formulation embodiment. The method steps are repeated as appropriate. By “mechanically debride or mechanically debriding” is meant that tissue at and around a wound that is lacerated or devitalized or contaminated is removed with a mechanical device typically used in surgery such as a scissors or a scalpel or other surgical instrument.

The mechanical debridement includes scraping and excising tissue from a wound in order to reach healthy, viable tissue. In some instances, debridement includes cutting through edges and base of a wound in order to reach healthy tissue. The debridement is used with a scalpel, ring curette, scissors and forceps.

### EXAMPLE

### Material and Methods:

A prospective, observational study took place at a private medical practice in Blaine, MIN from Sep. 12, 2022 through Nov. 23, 2022. This study adhered to the principles embodied in the Declaration of Helsinki. Adult patients with chronic DFUs or VLUs over 30 days duration were considered eligible for the study, provided that they gave their written informed consent. The only exclusion criterion was the presence of chronic critical limb ischemia (CCLI), defined as having have a transcutaneous oxygen measurement less than 40 mmHg.

The primary endpoint of this prospective, single-center study was wound pH measured by a pH meter at baseline, postdebridement, postapplication (in the RDT group only), and on Day 1, Day 7, and Day 14. The secondary endpoint was the percentage volume reduction (PVR) on Day 1, Day 7, and Day 14. All wounds underwent sharp debridement. In the RDT group, RDT was applied with a gloved finger to the wound bed for 30-60 seconds and then rinsed with normal, sterile saline. Both groups continued standard of care, including offloading diabetic foot ulcers (DFUs) and multilayer compression of venous leg ulcers (VLUs).

The RDT, regenerative debridement technology agent, employed in the study had a hydroxybenzenesulfonic acid concentration of 35.3% by weight; a hydroxymethoxybenzenesufonic acid concentration of 23.3% by weight; sulfuric acid concentration of 28% by weight; water concentration of 10.2% by weight; a silica gel concentration of 3.3% by weight and a colorant of 0.05% by weight. The viscosity of the RDT was more than 1460 cPs at 20 degrees Centigrade. The density was more than 1.6 gm/mL at 20 degrees C. The RDT was applied with a syringe.

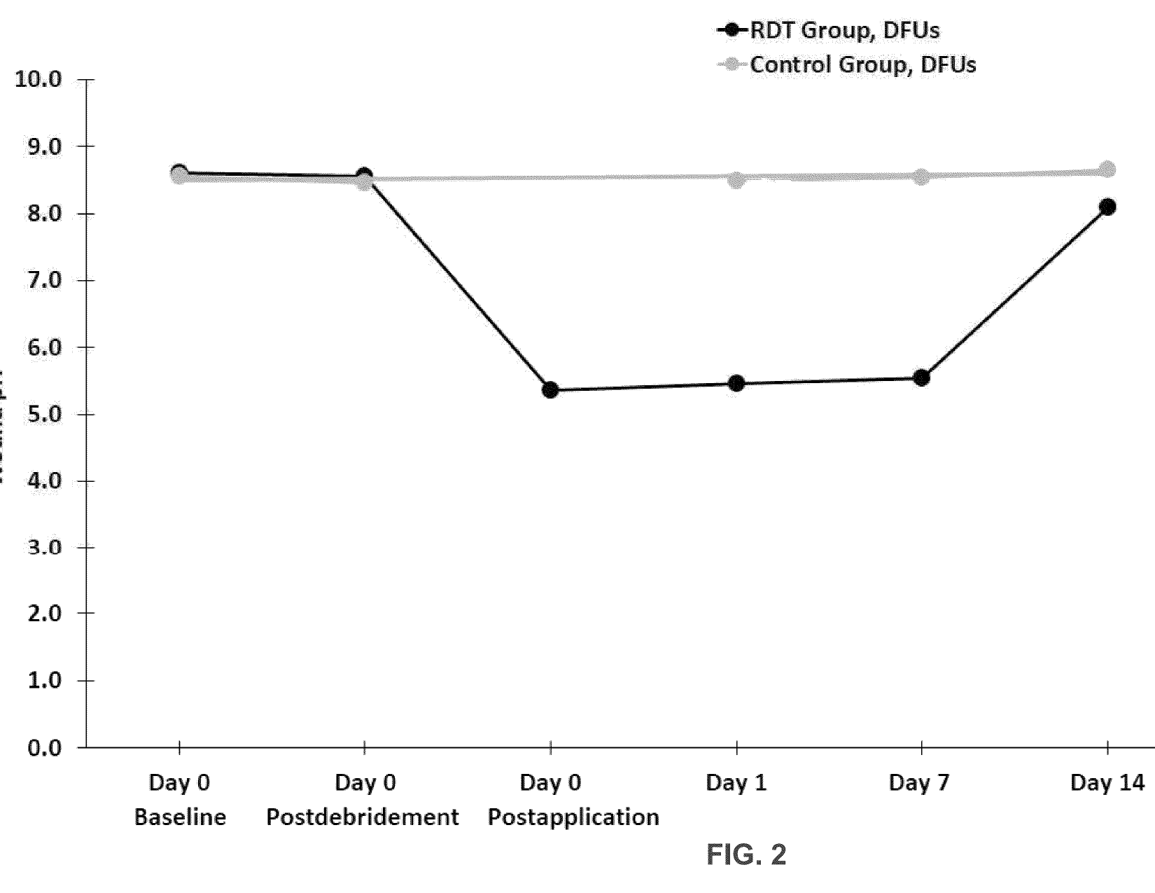

This study investigated the effect of RDT, a nonbiologic debridement agent, as a potential therapeutic pH modulator in the wound bed of chronic DFUs and VLUs treated with RDT, a regenerative debridement technology agent, and standard of care compared to standard of care alone. Results are shown in FIG. 1 and FIG. 2.

Thirty-eight patients [mean (SD) age: 67(8) years] participated, including 28 in the RDT group and 10 controls Immediately after RDT application, the mean (SD) pH decreased in the RDT group from 8.8 (0.3) to 5.4 (0.2); the acidic pH was maintained through Day 7 The control group had an alkaline state throughout the study. By Day 14, the mean (SD) PVR in the RDT group was 70% (6%) vs 18% (7%) among controls.

The primary endpoint was wound pH collected at baseline, immediately after sharp debridement, 30-60 seconds after RDT application (in the RDT group only), on Day 1, Day 7, and on Day 14 The secondary endpoint was the percentage volume reduction (PVR) on Day 1, Day 7, and Day 14. There was no sample size calculation performed. After enrolling 28 subjects with DFUs or VLUs into the RDT group, 5 patients with DFUs and 5 patients with VLUs were next enrolled into the control group (n=10 total). The control group served to provide exploratory comparative data, but the study was not powered to detect a significant difference between groups Wound volume was also measured using a standard ruler. Standard of care for all DFUs included applying a hydrogel dressing with a nonadherent dressing and gauze to the wound and offloading the index limb with a surgical rocker sole shoe or controlled ankle motion walker. All VLUs received an alginate dressing, a nonadherent dressing, and a multilayer compression system providing 35-40 mmHg. Eligible patients who provided their informed consent additionally underwent the study procedure at the initial visit

### Results:

There were 39 patients screened for eligibility in this study; only 1 patient (2.6%) was ineligible due to having CCLI. The remaining 38 patients (with 18 DFUs and 20 VLUs) were enrolled into the study. The first 28 patients (with 13 DFUs and 15 VLUs) were allocated to the RDT group. The last 10 patients (with 5 DFUs and 5 VLUs) were allocated to the control group. The mean (SD) age of all patients was 67 (8) years, and 15 (40%) were male (Table 1). This patient population had a mean (SD) number of 3.7 (1.1) serious comorbidities, and more than 75% had hypertension Patient characteristics were similar across groups.

All DFUs were Wagner 2 ulcers located on the plantar foot. All VLUs were full-thickness ulcers located on the distal leg, medial gator region (Table 2). The mean (SD) wound duration was 165 (133) days. The mean (S) baseline volume was 1,124 (991) mm³. Control wounds had a considerably longer duration than the wounds in the RDT group, but the RDT wounds were markedly larger in volume than the controls. The mean (SD) baseline pH was an alkaline 8.8 (0.4) and similar between groups, with control VLUs only having a larger pH than those in the RDT group (9.4 (0.3) vs 8.9 (0.3), respectively (Table 2).

All patients completed the study and received their allocated treatment. There were no missing data. On Day 0, immediately after RDT application, the mean (SD) pH decreased dramatically to 5.4 (0.2) (Table 3, FIG. 1). The acidic pH was maintained in the RDT group through Day 7, by Day 14, the RDT wounds returned to an alkaline state [8 (04)]. Wound pH in the control group remained in an alkaline state throughout the study duration and varied minimally in a linear fashion. These trends were observed for both DFUs and VLUs (Table 3, FIGS. 2 and 3). FIG. 4 depicts a case of a patient with a DFU (Patient No. 11) receiving his RDT application, with the formation of the coagulum noticeable in FIGS. 4B and 4C.

By Day 7, accelerated healing was observed in the RDT group, with the mean (SD) PVR almost double that of the controls [23% (5%) vs 12% (5%)] (Table 3). By Day 14, the mean (SD) PVR in the RDT group was an impressive 70% (6%) compared to only 18% (7%) in the control group. The PVR trends are consistent for all wounds, DFUs, and VLUs (FIGS. 5-7).

### Conclusions:

Directly after sharp debridement, there was no reduction of wound bed pH of any significance
Immediately following a single application, post sharp debridement, of RDT, mean wound pH decreased from an alkaline 8.7 to an acidic 5.6, which was sustained for at least 1 week post application in the RDT group, while wounds treated with standard of care alone remained in an alkaline state (with a pH≥8.3) until the end of study.
After 1 week, accelerated healing was observed in the RDT group, with the mean PVR almost double that of the controls (23% vs 12%).
Two weeks after RDT application, wounds in the RDT were experiencing rapid closure, with an impressive mean PVR of 70% compared to only 18% in the control group.
A single application of RDT accelerated wound healing by immediately decreasing wound pH to an acidic state for at least 1 week, allowing the wound to proceed with the normal healing trajectory, as evidenced by rapid closure rates after 2 weeks.

Since the invention disclosed herein may be embodied in other specific forms without departing from the spirit or general characteristics thereof, some of which forms have been indicated, the embodiments described herein are to be considered in all respects illustrative and not restrictive. The scope of the invention is to be indicated by the appended claims, rather than by the foregoing description, and all changes, which come within the meaning and range of equivalency of the claims, are intended to be embraced therein.

What is claimed is:

1. A method of promoting healing of tissue that has been wounded, comprising
Applying a formulation that includes phenolsulfonic acid and either guaiacolsulfonic acid or benzenesulfonic acid to tissue that has been wounded; normalizing pH of the tissue that has been wounded; and
Re-applying the formulation that includes phenolsulfonic acid and either guaiacolsulfonic acid or benzenesulfonic acid to the tissue that has been wounded over a time period effective to close a cut or break in the tissue that has been wounded and accelerating the healing the tissue that has been wounded compared to wounded tissue that has not been treated with the formulation that includes phenolsulfonic acid and either guaiacolsulfonic acid or benzenesulfonic acid.

2. The method of claim 1 wherein the formulation that includes phenolsulfonic acid and either guaiacolsulfonic acid or benzenesulfonic acid is reapplied to the tissue that has been wounded every seven days until the cut or break in the tissue that has been wounded has closed.

3. The method of claim 1, further comprising debriding necrotic tissue that has been wounded and sloughing the tissue that has been debrided, and
debriding planktonic bacteria from the tissue that has been wounded.

4. The method of claim 1, further comprising exposing the debrided tissue to low frequency ultrasound.

5. The method of claim 1, further comprising exposing the debrided tissue to laser energy.

6. The method of claim 1, further comprising exposing the debrided tissue to electro-stimulation.

7. The method of claim 1, wherein the formulation is a gel.

8. A method of promoting healing of tissue of a surgical site, comprising
normalizing pH of an environment contacting tissue of the surgical site that has been wounded by an incision and by contacting the tissue with a formulation that includes sulfonic acid and either guaiacolsulfonic acid or benzenesulfonic acid and re-contacting the tissue until the incision of the surgical site closes.

9. The method of claim 8 wherein the formulation that includes phenolsulfonic acid and either guaiacolsulfonic acid or benzenesulfonic acid is reapplied to the tissue that has been wounded by incision every seven days until the incision in the tissue that has been wounded has closed.

10. The method of claim 8, further comprising debriding necrotic tissue that has been wounded and sloughing the tissue that has been debrided; and
debriding planktonic bacteria from the tissue that has been wounded by incision.

11. The method of claim 8, further comprising exposing the debrided tissue to low frequency ultrasound.

12. The method of claim 8, further comprising exposing the debrided tissue to laser energy.

13. The method of claim 8, further comprising exposing the debrided tissue to electro-stimulation.

14. The method of claim 8, wherein the formulation is a gel.

15. A method of promoting healing of an acute wound, comprising:
normalizing pH of an environment contacting tissue of the acute wound and by contacting the tissue with a formulation that includes sulfonic acid and either guaiacolsulfonic acid or benzenesulfonic acid and re-contacting the tissue until the acute wound closes.

16. The method of claim 15 wherein the formulation that includes phenolsulfonic acid and either guaiacolsulfonic acid or benzenesulfonic acid is reapplied to the tissue that has been wounded every seven days until the wound has closed.

17. The method of claim 15, further comprising debriding necrotic tissue that has been wounded and sloughing the tissue that has been debrided; and
debriding planktonic bacteria from the tissue that has been wounded.

18. The method of claim 15, further comprising exposing the debrided tissue to low frequency ultrasound.

19. The method of claim 15, further comprising exposing the debrided tissue to laser energy.

20. The method of claim 15, further comprising exposing the debrided tissue to electro-stimulation.
